(19) United States
(12) Patent Application Publication
(10) Pub. No.: US 2024/0410884 A1
(43) Pub. Date: Dec. 12, 2024

(54) BIOLOGICAL DETECTION CHIP

(71) Applicant: DARWIN PRECISIONS CORPORATION, Hsinchu County (TW)

(72) Inventors: Yu-Hsin Wu, Hsinchu County (TW); Pang-Chun Liu, Hsinchu County (TW); Jui-Hua Lin, Hsinchu County (TW)

(21) Appl. No.: 18/534,758

(22) Filed: Dec. 11, 2023

(30) Foreign Application Priority Data
Jun. 9, 2023 (TW) 112121682

(51) Int. Cl. G01N 33/543

(52) U.S. Cl. CPC G01N 33/54346; G01N 33/563; G01N 33/56983

(57) ABSTRACT

A biological detection chip includes a base, multiple microstructures, a conducting layer, and a nano metal layer, wherein the base is provided with a surface; the multiple microstructures are arranged on the surface; and each of the microstructures protrudes out of the surface, and is provided with a first inclined plane and a second inclined plane inclining towards each other. The conducting layer covers the surface and the multiple microstructures, and the nano metal layer covers the conducting layer. The biological detection chip may increase the number of antibodies attached to the nano metal layer, so that the detection efficiency is improved.

### CROSS-REFERENCE TO RELATED APPLICATION

This application claims the priority benefit of Taiwan Application No. 112121682, filed on Jun. 9, 2023. The entirety of the above-mentioned patent application is hereby incorporated by reference herein and made a part of this specification.

### FIELD OF THE INVENTION

The present invention relates to a detection chip, and particularly relates to a biological detection chip.

### BACKGROUND OF THE INVENTION

Medical examinations for estimating the health condition and the physiological function can be also applied in the processes of diagnosing, treating and tracing diseases, and the like. The way to detect viruses includes enzyme linked immunosorbent assay (ELISA) and polymerase chain reaction (PCR). However, the above methods require professionals to operate expensive equipment and feature long reaction time. Therefore, a biological detection chip emerges as the times require. The biological detection chip features small size, simple operation, rapid reaction and low cost, so it is widely applied to detecting different viruses. A biological detection chip includes nano metal stably attached to the surface. Antibodies are attached to the nano metal. The biological detection chip grabs viruses by means of specificity of antigens towards antibodies. Moreover, as the viruses attached to the antibodies have the characteristic of increasing electric resistance, the change of the current value is measured electrochemically to determine whether there are viruses or not. However, the known biological detection chip has a limited number of attached antibodies, so the detection efficiency is poor.

### SUMMARY OF THE INVENTION

The present invention provides a biological detection chip which may increase the number of antibodies attached, so that the detection efficiency is improved.

The biological detection chip provided by the present invention includes a base, multiple microstructures, a conducting layer, and a nano metal layer, wherein the base is provided with a surface; the multiple microstructures are arranged on the surface; and each of the microstructures protrudes out of the surface, and is provided with a first inclined plane and a second inclined plane inclining towards each other. The conducting layer covers the surface and the multiple microstructures, and the nano metal layer covers the conducting layer.

In an embodiment of the present invention, there is a 40-60-degree included angle between the first inclined plane and the second inclined plane.

In an embodiment of the present invention, the first inclined plane is connected to the second inclined plane, so that each of the microstructures forms a triangular prism.

In an embodiment of the present invention, a top edge is formed at a connection between the first inclined plane and the second inclined plane, and a distance between the top edge and the surface is 2-3 μm.

In an embodiment of the present invention, the microstructures include multiple first microstructures and multiple second microstructures; the first microstructures are arranged in multiple rows, and the second microstructures are arranged in multiple columns; and the rows and columns intersect in a latticed shape.

In an embodiment of the present invention, a distance between the rows is 2-4 μm, and a distance between the columns is 2-4 μm.

In an embodiment of the present invention, a material of the conducting layer includes gold.

In an embodiment of the present invention, a material of the nano metal layer includes colloidal nanogold.

In an embodiment of the present invention, there is an adsorption layer between the conducting layer and the nano metal layer.

In an embodiment of the present invention, the biological detection chip further includes an electrode electrically connected to the conducting layer.

As the biological detection chip provided by the present invention adopts the microstructures which protrude out of the surface and have the first inclined planes and the second inclined planes, the nano metal layer covers the surface uniformly, so that the number of the antibodies attached increases, and the detection efficiency is improved.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

The present invention will become more readily apparent to those ordinarily skilled in the art after reviewing the following detailed description and accompanying drawings, in which:

FIG. 1 is a schematic diagram of a partial section of a biological detection chip in an embodiment of the present invention;

FIG. 2 is a partial three-dimensional schematic diagram of a base and multiple microstructures in an embodiment of the present invention;

FIG. 3 is a schematic diagram of a partial section of multiple microstructures in another embodiment of the present invention; and

FIG. 4 is a schematic diagram of a biological detection chip in an embodiment of the present invention.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

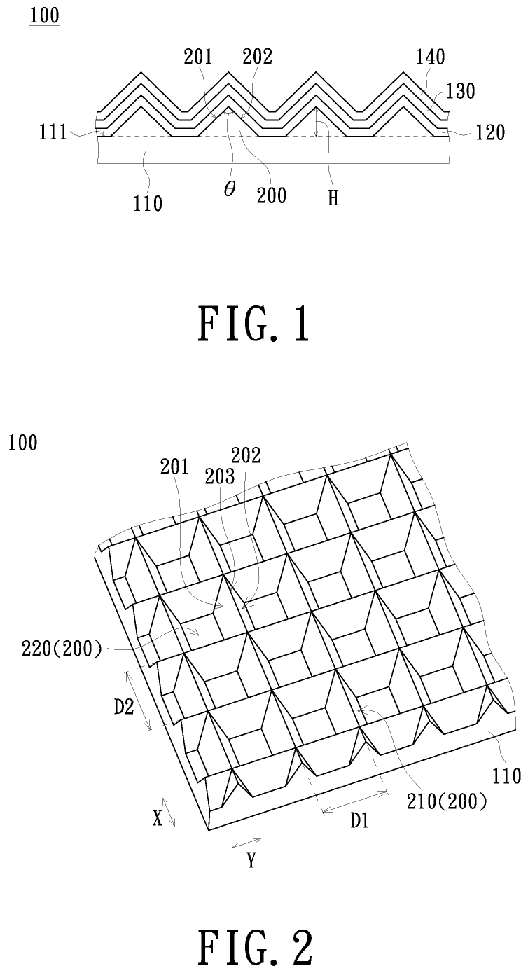

FIG. 1 is a schematic diagram of a partial section of a biological detection chip in an embodiment of the present invention. Referring to FIG. 1, the biological detection chip 100 provided by the embodiment includes a base 110, multiple microstructures 200, a conducting layer 120, and a nano metal layer 140, wherein the base 110 is provided with a surface 111; the multiple microstructures 200 are arranged on the surface 111. Each of the microstructures 200 protrudes out of the surface 111 and is provided with a first inclined plane 201 and a second inclined plane 202 inclining towards each other. The conducting layer 120 covers the surface 111 and the multiple microstructures 200, and the nano metal layer 140 covers the conducting layer 120. Specifically speaking, as the antibodies are attached to the nano metal on the nano metal layer 140, to increase the surface area of the nano metal layer 140 will contribute to improving the number of antibodies attached of the biological detection chip 100, so that the detection efficiency is improved.

Most microstructures in the prior art are semispherical (not shown in the drawings), which often have the problem that the nano metal layer is deposited unevenly. It is analyzed by the inventor that the problem is induced by the shape of the microstructures. Specifically speaking, as the conducting layer 120 covers the surface along the base 110 and the microstructures 200, and a common method for forming the nano metal layer 140 includes: dropping a liquid containing the nano metal to the conducting layer 120; drying the liquid; and depositing the nano metal on the conducting layer 120 to form the nano metal layer 140, the deposition effect of the nano metal layer 140 will be affected by the microstructures 200. With respect to the microstructures in the prior art, since the semispherical surface is a curved surface with great slope change, the surface approaching to the base is nearly perpendicular, so a gap is easily formed at a junction of the microstructure and the base after the liquid containing the nano metal is dropped, resulting in uneven deposition of the nano metal layer.

To solve the problem of uneven deposition, the first inclined planes 201 and the second inclined planes 202 of the microstructures 200 incline relative to the surface 111, respectively, which contributes to covering the surface 111 and the microstructures 200 completely with the liquid containing the nano metal to form the nano metal layer 140 covering the conducting layer 120 uniformly, so as to prevent the microstructures 200 close to the base 110 from interacting approximately perpendicularly on the surface 111 and prevent the gaps formed by the liquid containing the nano metal between the microstructures 200 and the surface 111. Thus, the problem of uneven covering of the nano metal layer 140 can be improved. In other words, because the microstructures 200 of the biological detection chip 100 in the embodiment are provided with the first inclined planes 201 and the second inclined planes 202, the nano metal layer 140 can cover the conducting layer 120 uniformly to increase the covering integrity, so that the number of the antibodies attached to the nano metal layer 140 increases, and the detection efficiency is improved. In addition, the slopes of the first inclined planes 201 are, for example, consistent, and the slopes of the second inclined planes 202 are, for example, consistent, which are not limited in the present invention. A material of the conducting layer 120 in the embodiment includes, for example, gold, and a material of the nano metal layer 140 includes, for example, colloidal nanogold. It is not specifically limited in the present invention.

To continue, the slopes of the first inclined plane 201 and the second inclined plane 202 of the microstructure 200 relative to the surface 111 will affect the deposition effect of the nano metal layer 140. For example, in a case where the first inclined plane 201 intersects with the surface 111 at a right angle approximately, a gap is easily formed when the nano metal layer 140 is deposited. Similarly, the second inclined plane 202 also has the same condition, which is not repeatedly described here. For example, there is a 40-60-degree included angle between the first inclined plane 201 and the second inclined plane 202 in the embodiment, so that the deposition effect of the nano metal layer 140 is better (i.e., the depositing uniformity is excellent), and meanwhile, it also has the advantage of increasing the surface area of the microstructures 200 and the nano metal layer 140. For example, the included angles between the first inclined plane 201 and the surface 111 and between the second inclined plane 202 and the surface 111 in the embodiment are equal, for example, between 60-70 degrees, which is not specifically limited in the present invention. In another embodiment of the present invention, the included angles between the first inclined plane 201 and the surface 111 and between the second inclined plane 202 and the surface 111 in the embodiment are, for example, different.

Continuously referring to FIG. 1, there is an adsorption layer 130 between the conducting layer 120 and the nano metal layer 140 in the embodiment. When the material of the nano metal layer 140 is, for example, the colloidal nanogold, it can be applied to mercaptan molecules and gold atoms to generate sulfur-gold bonding with strong bonding force, i.e., the adsorption layer 130 in the embodiment. Thus, the nano metal layer 140 can stably cover the conducting layer 120, which is not limited hereto.

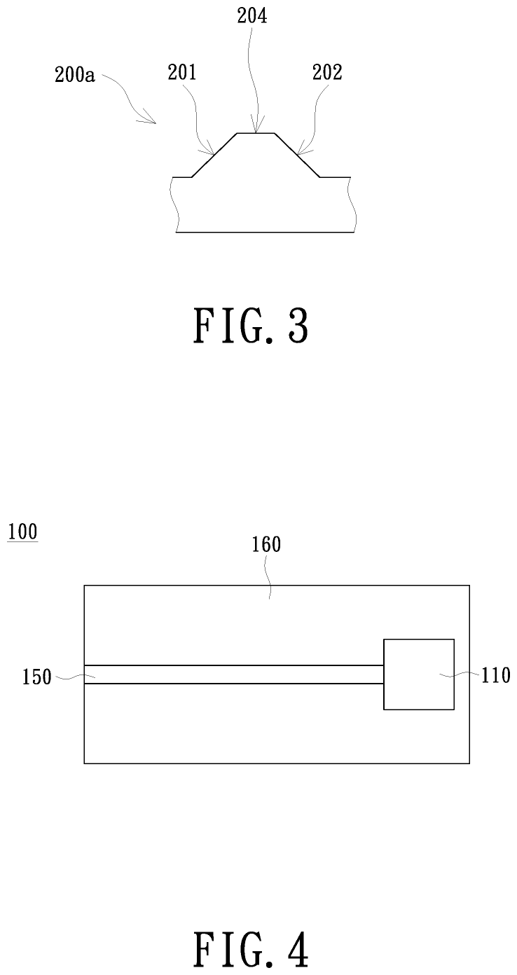

FIG. 2 is a partial three-dimensional schematic diagram of a base and multiple microstructures in an embodiment of the present invention. Referring to FIG. 1 and FIG. 2 together, the first inclined plane 201 and the second inclined plane 202 in the embodiment are, for example, connected, so that each of the microstructures 200 forms a triangular prism, i.e., the section of the microstructure 200 in the embodiment is, for example, triangular, wherein the inclined angles between the first inclined plane 201 and the surface 111 and between the second inclined plane 202 and the surface 111 are, for example, equal, and the section of the microstructure 200 is, for example, isosceles triangular, which is not limited hereto. In another embodiment, for example, a connecting surface 204 is further included between the first inclined plane 201 and the second inclined plane 202. As shown in FIG. 3, the microstructure 200a forms, for example, a quadrangular prism, for example, a trapezoidal column in the embodiment, which is not specifically limited in the present invention. Continuously referring to FIG. 2, a top edge 203 is, for example, formed at a connection between the first inclined plane 201 and the second inclined plane 202, and a distance H between the top edge 203 and the surface 111 is, for example, 2-3 μm, which is not limited in the present invention.

The multiple microstructures 200 in the embodiment can include multiple first microstructures 210 and multiple second microstructures 220. The multiple first microstructures 210 can be arranged in multiple rows, and each row is, for example, parallel to a first direction X, and the multiple second microstructures 220 can be arranged in multiple columns, and each column is, for example, parallel to a second direction Y, wherein the multiple rows and the multiple columns intersect, for example, in a latticed shape. Specifically speaking, the first microstructures 210 in the same row can be connected or disconnected to each other, and the second microstructures 220 in the same column can be connected or disconnected to each other. In an embodiment where the first microstructures 210 in the same row are connected to each other, the integral surface area of the microstructures 200 can increase, and similarly, in an embodiment where the second microstructures 220 in the same column are connected to each other, the integral surface area of the microstructures 200 can increase, too. To increase the integral surface area of the microstructures 200 will contribute to improving the surface area of the nano metal layer 140 and increasing the number of the antibodies attached thereto, so the detection efficiency can be improved. Furthermore, the first microstructures 210 and the second microstructures 220 in the embodiment intersect, for example, to form the lattice shapes (i.e., the rows and the columns intersect), so the integral surface area of the microstructures 200 can further increase, and the surface area of the nano metal layer 140 increases. Although the embodiment is disclosed above, the same column or the multiple microstructures 200 in the same column in another embodiment of the present invention may not be connected or may only be partially connected, or the first microstructures 210 may not intersect with the second microstructures 220, which is not specifically limited in the present invention.

On the other hand, because the first microstructures 210 in the embodiment are arranged, for example, in multiple rows, and the second microstructures 220 are arranged, for example, in multiple columns, in consideration of the characteristic of surface tension of the liquid, the distance D1 between the multiple rows in the embodiment is between 2 μm and 4 μm, and the distance D2 between the multiple columns is between 2 μm and 4 μm, which will contribute to filling the gaps between the rows and between the columns when the liquid containing the nano metal is dropped. Besides, it will further avoid a condition that the nano metal layer 140 cannot cover the conducting layer 120 evenly due to the liquid failing to fill the gaps as a result of surface tension because of a small the distance D1 or D2. It is worth mentioning that the size of the microstructures 200 in the embodiment can be in micron scale, which can improve the detection effect of the biological detection chip. Therefore, the microstructures 200 in the embodiment can be manufactured by precision finishing with cutlery. Compared with nanoscale microstructures manufactured by a semiconductor process in the prior art, the microstructures 200 in the embodiment have the advantages of low cost and high manufacturing efficiency, and the nano metal layer 140 can be deposited uniformly to increase its surface area, so that a better detection effect is achieved.

FIG. 4 is a schematic diagram of a biological detection chip in an embodiment of the present invention. Referring to FIG. 1 and FIG. 4, the biological detection chip 100 in the embodiment can further include an electrode 150 electrically connected to the conducting layer 120. Specifically speaking, the biological detection chip 100 can further include a basal plate 160, wherein the base 110 and the electrode 150 are arranged, for example, on the basal plate 160. The electrode 150 can provide a current, and the nano metal layer 140 covers the conducting layer 120. Therefore, when the current is conducted to the conducting layer 120, whether the antibodies attached to the nano metal layer 140 grab viruses can be determined by means of a numerical value of the current. The present invention does not specifically limit the component which provides the current as the electrode 150, and other components capable of providing current can be used as well.

In conclusion, as the biological detection chip in the embodiment adopts the microstructures which protrude out of the surface and have the first inclined planes and the second inclined planes, the nano metal layer covers the surface uniformly, so that the number of the antibodies attached increases, and the detection efficiency is improved.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A biological detection chip, comprising:
a base provided with a surface;
multiple microstructures arranged on the surface, wherein each of the microstructures protrudes out of the surface, and is provided with a first inclined plane and a second inclined plane inclining towards each other;
a conducting layer covering the surface and the microstructures; and
a nano metal layer covering the conducting layer.

2. The biological detection chip according to claim 1, wherein there is a 40-60-degree included angle between the first inclined plane and the second inclined plane.

3. The biological detection chip according to claim 1, wherein the first inclined plane is connected to the second inclined plane, so that each of the microstructures forms a triangular prism.

4. The biological detection chip according to claim 3, wherein a top edge is formed at a connection between the first inclined plane and the second inclined plane, and a distance between the top edge and the surface is 2-3 μm.

5. The biological detection chip according to claim 1, wherein the microstructures comprise multiple first microstructures and multiple second microstructures; the first microstructures are arranged in multiple rows, and the second microstructures are arranged in multiple columns; and the rows and columns intersect in a latticed shape.

6. The biological detection chip according to claim 5, wherein a distance between the rows is 2-4 μm, and a distance between the columns is 2-4 μm.

7. The biological detection chip according to claim 1, wherein a material of the conducting layer comprises gold.

8. The biological detection chip according to claim 1, wherein a material of the nano metal layer comprises colloidal nanogold.

9. The biological detection chip according to claim 1, wherein there is an adsorption layer between the conducting layer and the nano metal layer.

10. The biological detection chip according to claim 1, further comprising an electrode electrically connected to the conducting layer.
